(19) United States
(12) Patent Application Publication
(10) Pub. No.: US 2010/0130958 A1
(43) Pub. Date: May 27, 2010

(54) Device and Methods for Subcutaneous Delivery of High Viscosity Fluids

(76) Inventors: David Kang, Encinitas, CA (US); Monica Zepeda, San Diego, CA (US)

Correspondence Address: DLA PIPER LLP (US), 4365 EXECUTIVE DRIVE, SUITE 1100, SAN DIEGO, CA, 92121-2133, US

(21) Appl. No.: 12/324,562

(22) Filed: Nov. 26, 2008

(51) Int. Cl. A61M 5/158

(52) U.S. Cl. 604/506; 604/239

(57) ABSTRACT

The present invention provides infusion devices and methods for subcutaneous delivery of high viscosity fluids. The devices and methods described are innovative subcutaneous infusion systems that allow for the rapid delivery of viscous therapeutic fluids while avoiding adverse side effects and promoting patient comfort. The devices and methods described are also useful for rapid delivery of high volumes of therapeutic fluids.

### BACKGROUND OF THE INVENTION

1. Field of the Invention

The invention relates generally to the field of subcutaneous infusion of fluids and more particularly to infusion sets and methods for subcutaneous delivery of high viscosity fluid therapeutics.

### BACKGROUND INFORMATION

A standard method for the delivery of therapeutic fluids into a patient's body is by injection or infusion. Intravenous delivery of therapeutic fluids involves administration of the fluid directly into the patient's circulatory bloodstream by puncturing the patient's vein, for example, with an injection needle. Yearly, in U.S. hospitals alone, practitioners place well over 25 million intravenous catheters. Regardless of the fact that intravenous placement requires skill, patients often endure failed attempts due to difficult venous access associated with skin pigmentation, vein sclerosis, fragility, collapse, or obesity or vasovagal reaction due to stress. Furthermore, catheter complications include malfunction, thrombosis, infection, and extravasation, which decrease systemic access and increase the cost of care.

Alternatively, therapeutic fluids may be administered directly into subcutaneous tissue by insertion of a needle into the skin to traverse the epidermal and dermal layers of the skin to deliver fluid directly to the subcutaneous tissue. Subcutaneous delivery of therapeutics avoids many intravenous complications and costs. For example, results from two recent studies indicated that the cost of cannulae is reduced by approximately two thirds with subcutaneous versus intravenous hydration. Because subcutaneous catheter placement requires less skill, patients and caregivers can develop expertise. subcutaneous catheter sites minimize bleeding and thrombosis risk. Additionally, if infection occurs, it is typically localized and there are many placement sites for needle placement including, for example, the upper chest, arms, upper back, abdomen, and thighs.

Subcutaneous tissue or subcutis is the layer of loose connective tissue directly underlying the dermis. It is mainly composed of adipose tissue and its thickness depends on the amount of fat present, which is largely determined by the area of the body and the individual person. Subcutaneously injected therapeutics must pass through the loose connective tissue of the skin in order to reach their intended targets, such as underlying blood and lymphatic vessels.

The subcutaneous space consists of collagen surrounded by connective tissue including hyaluronan, a glycosaminoglycan. Hyaluronan is found in mammals predominantly in connective tissues, skin, cartilage, and in synovial fluid. Hyaluronan is also the main constituent of the vitreous of the eye. Hyaluronan, the main substrate for hyaluronidase, is a repeating disaccharide of [GlcNAc.beta. 1-4GlcUA.beta. 1-3]ₙ that exists in vivo as a high molecular weight linear polysaccharide. Degradation of hyaluronan by hyaluronidase is accomplished by either cleavage at β-N-acetyl-hexosamine-[1→4]-glycosidic bonds or cleavage at β-gluconorate-[1→3]-N-acetylglucosamine bonds. Hyaluronan's highly viscous gel-like consistency is a major barrier to subcutaneous diffusion.

Generally, in order to subcutaneously dispense a fluid from an external source to a patient, the proximal end of a hollow needle is inserted through the skin of the patient, thereby providing a passageway to the desired subcutaneous injection location under the skin of the patient. The distal end of the hollow needle located externally of the epidermis of the patient is connected to or in fluid communication with a reservoir containing the therapeutic fluid. To facilitate delivery of the fluid, pressure is typically applied to the reservoir to move the fluid through the reservoir and hollow needle to the subcutaneous tissue.

Subcutaneous infusion of therapeutic fluids, such as biologics, offers benefits over other methods of transdermal delivery. However, due to the complex, three-dimensional structure of the subcutaneous layer, due in part to the presence of hyaluronan, the type and quantity of therapeutics that can be administered by subcutaneous infusion is limited. For example, subcutaneous infusion of highly viscous fluid therapeutics offers particular challenges. One particular challenge is achieving appropriate flow rates of therapeutics of a viscous nature during infusion such that the therapeutic retains efficacy while avoiding harmful side effects, such as unnecessary pain or edema.

Unfortunately, methods and devices for subcutaneous delivery of viscous fluid therapeutics to the subcutaneous tissue have not yet been described that allow for the administration of a large variety of viscous fluid therapeutics as well as administration of large volumes of fluid.

### SUMMARY OF THE INVENTION

The present invention is based in part on the discovery of innovative subcutaneous infusion systems that allow for the effective delivery of viscous therapeutic fluids as well as delivery of large volumes while avoiding adverse side effects and promoting patient comfort.

Accordingly, in one aspect, the present invention provides a subcutaneous infusion system for delivery of viscous therapeutic fluids. The system includes an infusion needle and a dispensing device. The infusion needle is hollow and includes a shaft having an internal duct of unvarying diameter defining a fluid pathway between openings at distal and proximal ends of the needle. The infusion system further includes a hub surrounding or coupled to the outside of the needle having a bore therethrough that is in fluid communication with the needle duct.

In various embodiments, the needle may be a 24 to 27 Ga needle having a straight bore and length of about 4 to 6 mm measured from the hub to the proximal opening of the needle. The drug dispensing device may be secured at the hub in fluid communication with the distal opening of the needle and includes about 3 to 100 mls of a therapeutic fluid composition. The system is configured for subcutaneous delivery of the therapeutic fluid composition at a flow rate of about 1-20 mls/min. In one embodiments, the needle and delivery device are in fluid communication via tubing connecting the distal opening of the needle to the drug dispensing device. Alternatively, in another embodiment, the dispensing device is in direct fluid communication with the needle. Alternatively, in another embodiment, the dispensing device is in fluid communication with the needle, via a chamber disposed between the fluid reservoir and the distal opening of the needle.

In another aspect, the invention provides a method for subcutaneous delivery of a high viscosity therapeutic fluid composition to a subject. The method includes administering to the subject a therapeutic fluid using an infusion device described herein, wherein the therapeutic fluid is administered at a flow rate of about 1-20 ml/min. In an exemplary embodiment, the therapeutic fluid is administered at a flow rate of about 3-5 ml/min. In another embodiment, the therapeutic fluid has a viscosity of about 10 to 20 cP. In various embodiments, the fluid is administered to the subcutaneous tissue at any location of the body, such as but not limited to the abdomen, arm or thigh, gluteus region, leg, back, chest. In yet another embodiment, the therapeutic fluid includes a hyaluronidase enzyme, such as human hyaluronidase to facilitate dispersion of the therapeutic fluid within the subcutaneous tissue.

In yet another aspect, the invention provides a method for rapid subcutaneous delivery of a high volume of a therapeutic fluid to a subject in need thereof. The method includes administering to the subject a therapeutic fluid using a subcutaneous infusion system as described herein configured with an 18 Ga needle, wherein the fluid is administered at a flow rate greater than about 20 ml/min.

### BRIEF DESCRIPTION OF THE DRAWINGS

FIG. 1 is a graphical representation showing epidermal-dermal thickness of the abdomen, arm and thigh of 31 human subjects.

FIG. 2 is a graphical representation showing fascial thickness of the abdomen, arm and thigh of 31 human subjects.

FIG. 3 is a schematic of the system used to conduct flow analysis using various infusion set embodiments.

FIG. 4 is a pictorial representation showing the needle component of one embodiment of the present invention. The needle (1), having a shaft with distal (3) and proximal openings is connected to tubing (4). Integral with the shaft of the needle is the hub (2).

FIG. 5 is a pictorial representation showing one embodiment of the infusion set including a needle (31) in fluid communication with a dispensing device (32) via an injection chamber (25).

FIG. 6 is a graphical representation of inline pressures generated by the various infusion sets represented in Table 15. Solutions containing different percentages of human IgG were used at a flow rate of 4.0 ml/min. The top set of three lines presented in the plot correspond to the infusion sets having 27 Ga needles. The middle set of three lines presented in the plot correspond to the infusion sets having 25 Ga needles. The bottom set of 2 lines presented in the plot correspond to the infusion sets having 24 Ga needles.

FIG. 7 is a graphical representation of inline pressures generated by the various infusion sets represented in Table 15. Solutions containing different percentages of human IgG were used at a flow rate of 1.7 ml/min. The top set of three lines presented in the plot correspond to the infusion sets having 27 Ga needles. The middle set of three lines presented in the plot correspond to the infusion sets having 25 Ga needles. The bottom set of 2 lines presented in the plot correspond to the infusion sets having 24 Ga needles.

### DETAILED DESCRIPTION OF THE INVENTION

The invention relates generally to the field of subcutaneous infusion of fluids and more particularly to infusion sets and methods for subcutaneous delivery of high viscosity fluids as well as delivery of high volumes of fluid in ranges much greater than the traditional range of 1-2 ml.

The present invention is based, in part on the discovery of innovative subcutaneous infusion systems that allow for the effective delivery of viscous therapeutic fluids while avoiding adverse side effects and promoting patient comfort.

Before the present devices and methods are described, it is to be understood that this invention is not limited to particular device designs, methods, and experimental conditions described, as such designs, methods, and conditions may vary. It is also to be understood that the terminology used herein is for purposes of describing particular embodiments only, and is not intended to be limiting, since the scope of the present invention will be limited only in the appended claims.

As used in this specification and the appended claims, the singular forms “a”, “an”, and “the” include plural references unless the context clearly dictates otherwise. Thus, for example, references to “the method” includes one or more methods, and/or steps of the type described herein which will become apparent to those persons skilled in the art upon reading this disclosure and so forth.

Unless defined otherwise, all technical and scientific terms used herein have the same meaning as commonly understood by one of ordinary skill in the art to which this invention belongs. Although any methods and materials similar or equivalent to those described herein can be used in the practice or testing of the invention, the preferred methods and materials are now described.

Accordingly, in one aspect, the present invention provides a subcutaneous infusion system for subcutaneous delivery of viscous therapeutic fluids. The system includes an infusion needle and a dispensing device. The infusion needle is hollow and includes a shaft having an internal duct of unvarying diameter defining a fluid pathway between openings at distal and proximal ends of the needle. The infusion system further includes a hub surrounding or coupled to the outside of the needle having a bore therethrough that is in fluid communication with the needle duct. In various embodiments, the needle may be a 24 to 27 Ga needle having a straight bore and length of about 4 to 6 mm measured from the skin side of the hub to the proximal tip of the needle.

The length of the needle from the hub determines the depth at which the needle may be inserted into the subcutaneous tissue. The soft tissue component of the connective tissue within the subcutaneous tissue below skin layers is referred to as fascia. Administration of a therapeutic fluid during subcutaneous infusion is to the subcutaneous layer lying beneath the skin layers. Thus the needle length from the skin side of the hub should be long enough to penetrate the skin layers while being an appropriate length to deliver fluid above or below the fascial layer or plane. In a preferred embodiment, the needle length is configured for administration above or below the fascial plane at about 1 to 2 standard deviations of the depth of the plane fascial plane. Preferably, the needle length is configured for administration

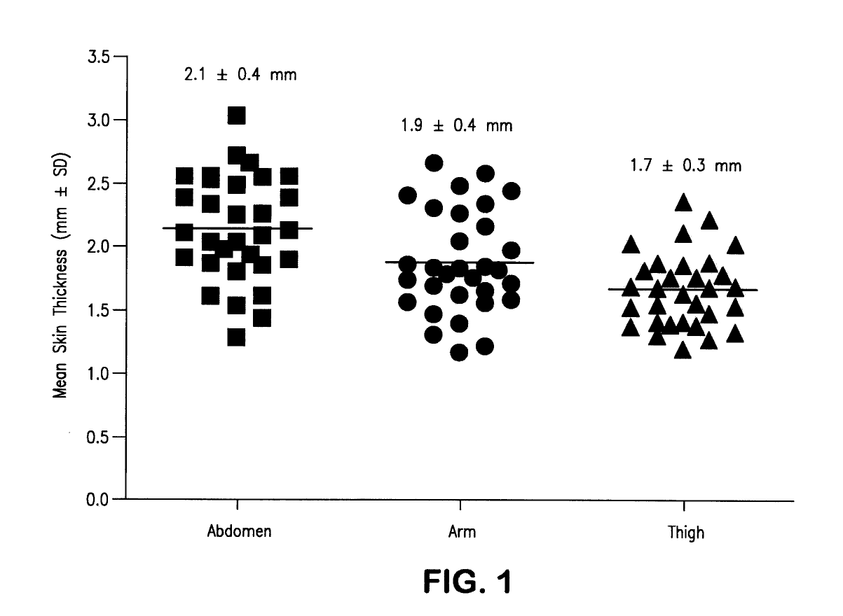

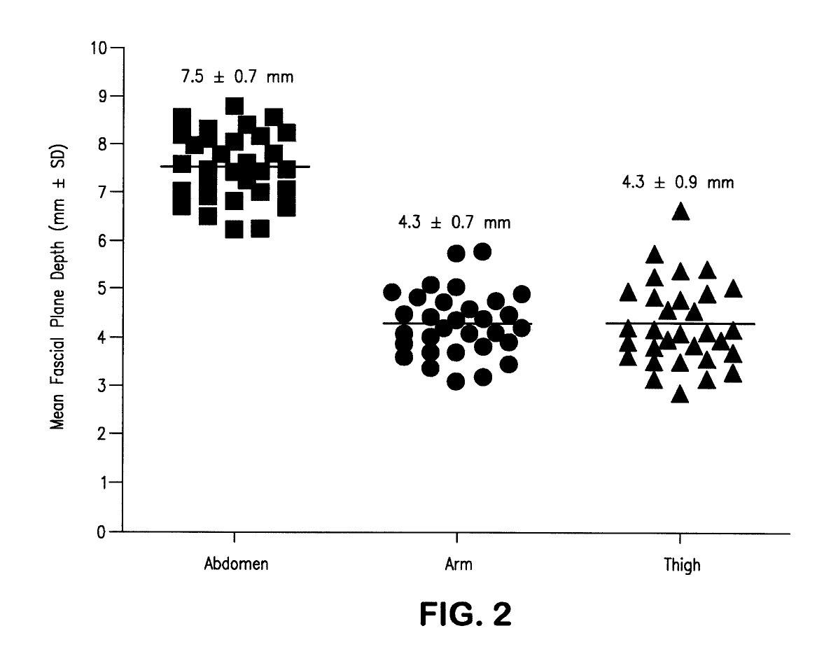

It has been established that skin and fascial layer thicknesses vary throughout different regions of the body as shown in FIGS. 1 and 2. For example, using ultrasound techniques, the average thickness of skin was established for the abdomen, arm and thigh as being 2.1, 1.9, and 1.7 mm respectively. Using similar methods, the average thickness of the underlying fascial layer was determined for the abdomen, arm and thigh as being 7.5, 4.3, and 4.3 mm respectively. Given the determined thicknesses of both the skin and fascial layers, the needle length from the skin side of the hub to the proximal tip of the needle for use in the infusion system described herein is preferably from about 4.0 to 6.0 mm. For example, the needle length from the skin side of the hub to the proximal tip of the needle is about 4.1, 4.2, 4.3, 4.4, 4.5, 4.5, 4.6, 4.7, 4.8, 4.9, 5.0, 5.1, 5.2, 5.3, 5.4, 5.5, 5.6, 5.7, 5.8, 5.9, or 6.0 mm in length.

The proximal tip of the needle may be of any shape or configuration suitable for piercing the skin layer to install the device into the subcutaneous tissue. For example, in various embodiments, the needle may have a beveled tip of between about 0 and 75 degrees. Alternatively, the needle may be introduced to a preexisting puncture in the skin. In such embodiments, the needle tip may be blunt. In various embodiments, the needle may be of any type suitable for the particular infusion application. By way of example and not intended to be limiting, the needle may be a non-coring, facil point, pencil point, trocar point, triangle point, or any other needle type known in the art. Additionally, the needle may have any number of side bored holes traversing the length of the needle on one or more sides of the needle (e.g., through holes). For example, in various preferred embodiments the needle is a non-coring side bore needle with one or more holes traversing the length of the needle. A needle may have any number greater than 1 additional opening or hole on the side of the shaft up to as many as can be added without impairing the rigidity or structural integrity of the needle cannula. One of skill in the art would understand that this would depend in part on characteristics such as the number, size, spacing, and geometry (e.g., shape of the opening and location on the shaft) of the openings and the type of needle used. Accordingly, the needle may have 1, 2, 3, 4, 5, 6, 7, 8, 9, 10, 50, or even greater than 100 holes (e.g., microperforated). Additionally, in various embodiments, the shape of the needle traveling from the proximal to the distal tip may be straight or bent between about 90 to 180 degrees (e.g., 90, 100, 110, 120, 130, 140, 150, 160, 170, to 180 degrees). In various embodiments, the bend may be bent abrupt or be a smooth “c” shaped bend. In preferred embodiments, the needle is straight or includes a smooth 90 degree bend.

In various embodiments, the drug dispensing device includes about 3 to 100 mls of a fluid therapeutic composition. For example, the dispensing device may include about 3 mls to about 5, 10, 15, 20, 25, 30, 35, 40, 54, 50, 55, 60, 65, 70, 75, 80, 85, 90, 95 or 100 mls. In an exemplary embodiment, the drug dispensing device includes about 3 to 15 mls of a fluid therapeutic composition. In another exemplary embodiment, the drug dispensing device includes about 10 to 15 mls of a fluid therapeutic composition

The device may be configured for delivery of a fluid therapeutic composition at various flow rates. It has been determined that the flow rate is determined, in part, by the dimensions of specific components of the of the infusion device. Accordingly, the device may be configured for delivery of fluid therapeutic compositions at a flow rate of about 1 to about 20 mls per minute. For example, the device may be configured for a flow rate of about 1, 2, 3, 4, 5, 6, 7, 8, 9, 10, 11, 12, 13, 14, 15, 16, 17, 18, 19 or 20 mls per minute.

The viscosity of a fluid therapeutic composition is dependent, in part, on the nature of the therapeutic, as well as the percent dilution of the fluid. While some therapeutic compositions may be diluted without compromising the efficacy of the composition, some compositions cannot be diluted without degrading the therapeutic, such as an active biological agent, or reducing effectiveness of the therapeutic due to reduced subcutaneous absorption. In various embodiments, the fluid therapeutic composition for subcutaneous delivery has a viscosity of about 1 cP to about 30 cP. For example, the viscosity may be about 5, 10, 15, 20, 25 or 30 cP. In an exemplary embodiment, the viscosity is about 10-20 cP.

One skilled in the art would realize that as the viscosity of the fluid being administered for a given devise configuration, higher flow rates are achieved. Accordingly, in various embodiments, the device may be configure for very high flow rates, using less viscous fluids. For example, with a fluid viscosity of less than about 5 (i.e., 1, 2, 3, 4, or 5), flow rates greater than about 10 ml/min (i.e., 10, 11, 12, 13, 14, 15, 16, 17, 18, 19, 20 or even greater than 20) may be achieved.

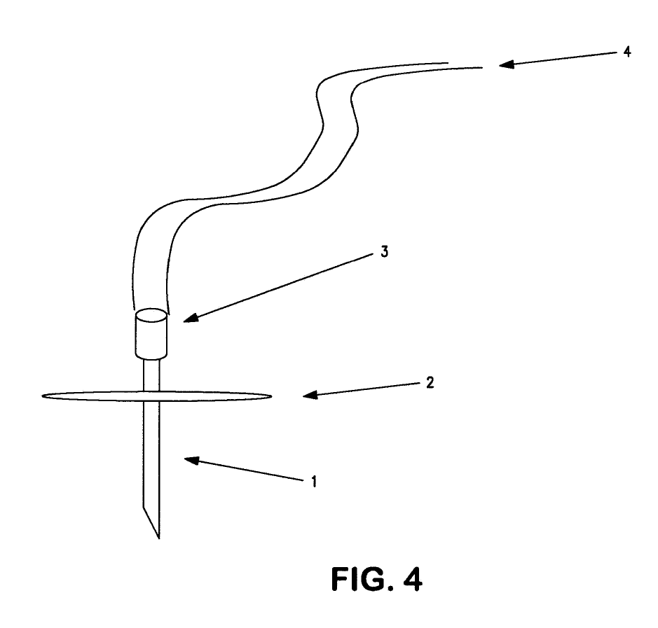

In various embodiments, the needle and delivery device are in fluid communication. Accordingly, in one embodiment, the needle and delivery device are in fluid communication via tubing connecting the distal opening of the needle to the drug dispensing device. FIG. 4 is a pictorial representation showing the needle component of one embodiment of the present invention. The needle (1), having a shaft with distal (3) and proximal openings is connected to tubing (4). Integral with the shaft of the needle is the hub (2).

The tubing is connected to the distal opening of the needle and the dispensing device using methods known in the art. Additionally, a number of different components may be disposed along the tubing or disposed at either end of the tubing, such as valves or the like. However, in an exemplary embodiment, any component interspersed along the tubing does not vary the internal diameter of the fluid pathway during active infusion. Additionally, the tubing length may be of any desired length from less than about 1 inch to about 20 inches. In an exemplary embodiment, the length of the tubing is about 6 to about 10 inches.

In addition to having a variable length, the tubing may have a variable inner diameter. For example, the inner diameter may be about 0.8 to about 1.4 mm. In an exemplary embodiment, the tubing has an internal diameter of about 1 mm to about 1.2 mm. For example, the inner diameter is preferably about 1, 1.1 or 1.2 mm.

In various embodiments, the tubing suitable for use with the infusion device of the present invention may be fabricated from any medical grade material, such as medical grade plastic or metal. In an exemplary embodiment, the tubing is a plastic polymer, such as, but not limited to polyethylene (PE), polyurethane (PUR), polyvinyl chloride (PVC), ethylene vinyl acetate (EVA), silicon, latex, teflon, nylon, or combinations thereof.

Alternatively, in another embodiment, the dispensing device is in direct fluid communication with the needle. For example, the dispensing device may be coupled directly to the needle.

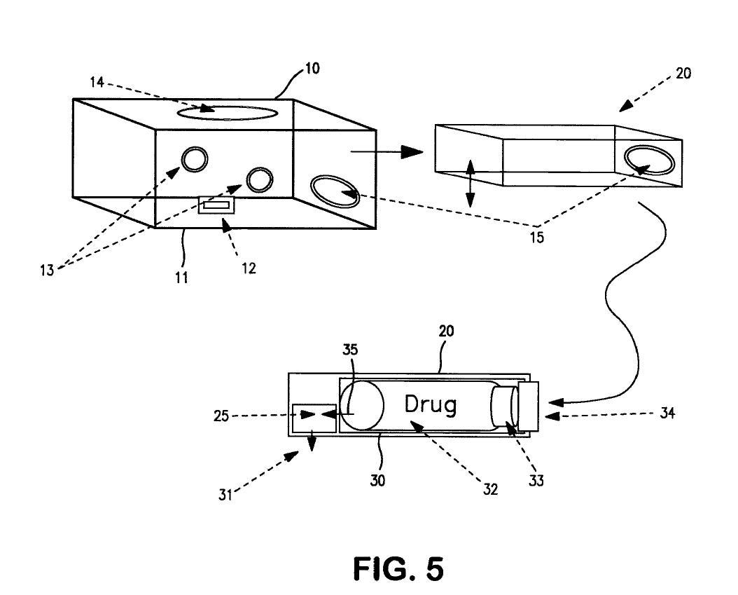

Alternatively, in another embodiment, the dispensing device is in fluid communication with the needle, via a chamber disposed between the fluid reservoir of the dispensing device and the distal opening of the needle. In such a configuration, the hub may be configured to serve as the housing for the dispensing device and the chamber. FIG. 5, shows one embodiment of the infusion device where the dispensing device is in fluid communication with the needle, via a chamber disposed between the fluid reservoir of the dispensing device and the distal opening of the needle. The infusion device includes an outer housing unit (10) which further includes a dispensing device (32) configured within a removable cartridge (20). The dispensing device is configured as a pre-filled drug reservoir including a therapeutic composition (32). The cartridge (20) further includes an injection chamber (25). In the embodiment shown in FIG. 5, the hub (11) is configured to serve as the housing (10) for the cartridge (20) including the dispensing device (32) and injection chamber (25). The injection chamber (25) is in fluid communication with the dispensing device (32) and the distal tip of the needle (31).

To operate the infusion system, the cartridge (20) is inserted into the housing (10). Various springs may be configured on cartridge (20) or within the housing (10) to hold the device within the housing in an inactivated position until the housing (10) is appropriately located on a subject via adhesive on the hub (11) and subsequently activated. Once the device is place at the desired infusion location, button (14) is pressed to lower the cartridge (20) within the outer housing (10) into a locked position in which needle (31) is inserted into the subject when button (14) is in the locked position. The dispensing device (32) is then activated by a spring or motor applying pressure to the plunger (34) pushing the pre-filled drug reservoir (32) of the cartridge (20) forward to pierce the reservoir at a point (35) allowing fluid to flow into the chamber (25) and through the injection needle (31) into the subject. In spring activated embodiments, pressing button (15) serves to move the pre-filled drug reservoir (32) within the cartridge to pierce the reservoir (32).

In both spring activated and motor activated embodiments, the spring or motor may be configured to apply sufficient pressure to the pre-filled drug reservoir (32) to impart the desired flow characteristics as described herein for viscous therapeutic compositions. Alternatively, the pre-filled drug reservoir (32) may be pressurized to achieve such flow characteristics.

The outer housing further includes, buttons (13) which when pressed unlock the cartridge (20) and allow the cartridge to move back into the upper position thus retracting the needle from the subject. Additionally, an indicator window (12) is included in the housing (10) to allow visible detection of when infusion of the therapeutic composition is complete by allowing visual detection of a colored plunger or strip (33) which travels along the cartridge (20) toward the injection chamber (25) as the drug is dispensed.

The infusion system may further include additional needles which are all inserted simultaneously upon pressing button (14). For example, in embodiments in which the dispensing device is in fluid communication with the needle as shown in FIG. 5, additional needles may be arranged adjacent to the needle (31) surrounded by the hub (11). The needles may be the same gauge or different gauges, but are preferably the same length as measured from the skin side of the hub (11) to the proximal openings of the needles. In an exemplary embodiment, the one or more needles have a length of about 4 mm to about 6 mm as measured from the hub to the proximal opening of the one or more needles. In various embodiments, the needles may be of any type known in the art as described herein. For example, in preferred embodiments the one or more needles are non-coring side bore needles with one or more holes traversing the length of the needle (e.g., having one or more side holes but no hole at the proximal end of the needle). Additionally, as discussed herein, the one or more needles may have any number greater than 1 additional opening or hole on the side of the shaft up to as many as can be added without impairing the rigidity or structural integrity of the needle cannula.

To operate embodiments in which the dispensing device is in fluid communication with the needle as shown in FIG. 5, the following exemplary sequence of operations may be employed. First, an adhesive backing disposed over the optional adhesive layer disposed on the hub (11) is removed and the infusion system is located over the site of injection. After the cartridge (20) is placed into the housing (10), button (14) is pressed to move the cartridge (20) into the locked lower position and also insert one or more needles (31) into the patient. Button (15) is then pressed to activate the infusion device by sliding the pre-filled drug reservoir (32) forward via a spring or actuator to pierce the reservoir (32) at a point (35) that allows the drug to flow out of the reservoir (32) into the injection chamber (25), through the infusion needle(s) (31) and into the patient. Once the assembly expels the entire injection volume, the colored plunger (33) becomes visible in the indicator window (12). Buttons (13) may then be pressed to ‘unlock’ the cartridge (20) from the locked position and retract the entire cartridge to the upper position along the retracting the needle(s) (31) from the patient.

In various aspects, the infusion systems of the present invention may utilize various dispensing devices to facilitate infusion of fluid. In various embodiments, the dispensing device has a fluid reservoir containing the therapeutic composition. A number of suitable delivery devices may be incorporated into the infusion system. For example, the dispensing device may include a syringe which optionally includes an automated motor for constant delivery of the fluid. Alternatively, the fluid reservoir of the dispensing device may be a pre-filled drug cartridge. The cartridge may be optionally pressurized or be coupled to an automated motor for constant delivery of the fluid.

In another aspect, the invention provides a method for subcutaneous delivery of a high viscosity therapeutic fluid composition to a subject in need thereof using the infusion system as described herein. The method includes administering to the subject a therapeutic fluid using an infusion device as described herein where the therapeutic fluid is administered at a flow rate of about 1-20 ml/min. For example, the fluid may be administered at a flow rate of about 1, 2, 3, 4, 5, 6, 7, 8, 9, 10, 11, 12, 13, 14, 15, 16, 17, 18, 19 or 20 mls per minute. In an exemplary embodiment, the fluid is administered at a flow rate of about 3-5 ml/min. Additionally, in various embodiments, the highly viscous fluid has a viscosity of about 1 cP to about 30 cP. For example, the viscosity may be about 5, 10, 15, 20, 25 or 30 cP. In an exemplary embodiment, the viscosity is about 10-20 cP.

In another aspect, the invention provides a method for rapid subcutaneous delivery of a high volume of a therapeutic fluid to a subject in need thereof. The method includes administering to the subject a therapeutic fluid using a subcutaneous infusion system as described herein configured with one or more 18 Ga needles. A device as described herein, is configured for subcutaneous delivery of fluid at high flow rates for use in emergency situations where a subject is in immediate need of therapeutic fluid. For example, situations where rapid delivery of a drug outweighs concerns over potential localized tissue damage. The fluid may be delivered at very high flow rates, such as greater than about 10, 20, 30, 40, 50 ml/min, or even greater.

As used herein, the terms “administration” or “administering” is intended to include an act of providing a therapeutic composition to a subject in need of treatment. The term “subcutaneous delivery” is intended to include administration of a therapeutic composition directly to the subcutaneous tissue of a subject. The term “subject” as used herein refers generally to humans, although as will be appreciated by those in the art, the subject may be an animal having similar skin and fascial layer characteristics, such as thickness.

The infusion system of the present invention may be configured for delivery of therapeutic compositions to any suitable area on a subject. However, as discussed herein, given the determined thicknesses of both the skin and fascial layers of the arm, abdomen and thigh, administration is preferably to these regions. However, due to the variability between thicknesses between individual subjects, administration may be to any area with the appropriate skin and fascial layer thicknesses.

As used herein, “therapeutic” composition or fluid is intended to include any composition relating to the treatment or prevention of disease or disorders. As will be appreciated by those skilled in the art, a therapeutic composition may include agents, such as but not limited to biologics, such as enzymes or antibodies; chemical compounds, such as organic molecules or small organic molecules; or the like. Additionally, by way of illustration and not intended to be limiting, the compositions may include nanoparticle or any therapeutics derived using molecular biological techniques such as genetic materials and/or recombinant biomolecules.

The therapeutic compositions may include one or more therapeutic agents. In various aspects, therapeutic agents may be combined to allow the agents to act synergistically in treatment or prevention. For example, therapeutic compositions may include combinations of therapeutic agents in which one agent facilitates dispersion, absorption, or uptake of another agent within the subcutaneous tissue.

As discussed above, subcutaneously injected therapeutics must pass through the interstitial matrix of the skin in order to reach their intended targets. This complex, three-dimensional structure limits the type and quantity of drugs that can be administered by local injection. However, coadministration of therapeutic agents with hyaluronidase enzyme promotes depolymerization of the viscoelastic component of the interstitial matrix resulting in increased dispersion of locally injected drugs, across a broad range of molecular weights without tissue distortion. Hyaluronidase enzyme increases infusion rates and the pattern and extent of appearance of locally injected drugs in systemic blood by enzymatic degradation of hyaluronan, a key component of the interstitial matrix. In specific hyaluronidase is known to change pharmacokinetic profiles of administered agents and significantly augment the absolute bioavailability of locally injected large protein therapeutics.

Accordingly, in various aspects, therapeutic compositions are coadministered with a hyaluronidase enzyme. In a preferred embodiment the hyaluronidase enzyme is a human hyaluronidase, recombinantly or naturally derived, where the subject is a human. Naturally derived human hyaluronidase enzymes are described in detail in U.S. Pat. Nos. 7,148,201, 7,105,330, 6,193,963, the entirety of which are incorporated herein by reference.

The following examples are provided to further illustrate the embodiments of the present invention, but are not intended to limit the scope of the invention. While they are typical of those that might be used, other procedures, methodologies, or techniques known to those skilled in the art may alternatively be used.

### EXAMPLE 1

### Flow Analysis of Infusion Sets

This example illustrates flow rate characteristics for a variety of infusion set designs.

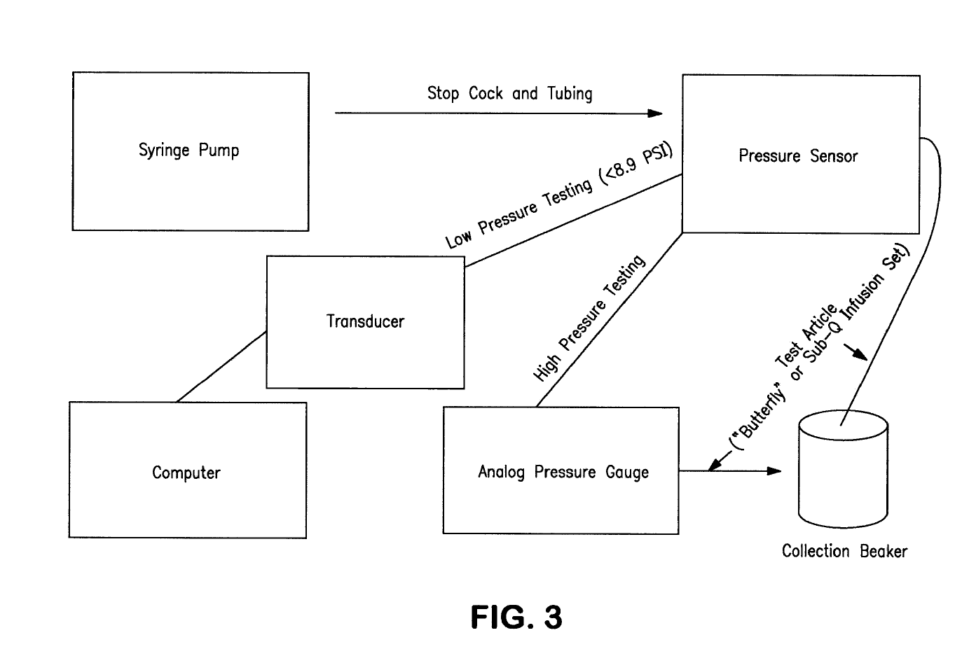

To conduct flow rate analysis of various infusion set configurations a system as shown in FIG. 3 was utilized. A mechanically driven syringe pump was coupled to a pressure sensor via tubing of varying lengths and inner diameters. To measure the inline high pressure an analog pressure gauge was disposed between the pressure sensor and a collection beaker. The inline low pressure was measured using a transducer coupled to the pressure sensor and interpreted by a computer.

Test regents utilized in the experiments included sterile water, 10%, 15%, 20% human IgG (Carimune M) solutions, and 10% and 20% human IgG (GammaGard™) solutions. Human IgG was incorporated into the test solutions to confer viscosity to the solution.

The critical parameters for infusion set design to achieve the desired flow rates and inline parameters were determined. As shown in the following tables, the following parameters were determined to affect inline pressure: 1) needle gauge; 2) extreme tubing length; 3) tubing inner diameter; and 4) needle shape. As shown in the following tables, the following parameters were determined to not affect inline pressure: 1) needle length; and 2) tubing length at shorter distance (less than about 12 inches). Test device names are specific to each individual table.

TABLE 1
The Effect of Needle Gauge on Inline Pressure

| | | Needle | | Tubing Inner | Mean Max | |
|---|---|---|---|---|---|---|
| Test | Needle | Length | Tubing | Diameter | Pressure | Standard |
| Device | Gauge (Ga) | (mm) | Length (in) | (mm) | (mmHg) | Deviation |
| A | 27 | 13 | 8 | ~1.0 | 729 | 0 |
| B | 25 | 13 | 8 | ~1.0 | 211 | 0.5 |

10% human IgG (Carimune ™) (n = 2); Flow rate = 4 mL/min

TABLE 2
The Effect of Needle Length on Inline Pressure

| | | Needle | | Tubing Inner | Mean Max | |
|---|---|---|---|---|---|---|
| Test | Needle | Length | Tubing | Diameter | Pressure | Standard |
| Device | Gauge (Ga) | (mm) | Length (in) | (mm) | (mmHg) | Deviation |
| A | 27 | 9 | 6 | 0.5 | 1448 | 0 |
| B | 27 | 4 | 6 | 0.5 | 1370 | 36.6 |

10% human IgG (Carimune ™) (n = 2); Flow rate = 4 mL/min

TABLE 3
The Effect of Extreme Tubing Length on Inline Pressure

| | | Needle | | Tubing Inner | Mean Max | |
|---|---|---|---|---|---|---|
| Test | Needle | Length | Tubing | Diameter | Pressure | Standard |
| Device | Gauge (Ga) | (mm) | Length (in) | (mm) | (mmHg) | Deviation |
| A | 27 | 9 | 6 | 0.5 | 620 | 0 |
| B | 27 | 9 | 42 | 0.5 | 1241 | 0 |

10% human IgG (Carimune ™) (n = 2); Flow rate = 1.7 mL/min

TABLE 4
The Effect of Tubing Inner Diameter on Inline Pressure

| | | Needle | | Tubing Inner | Mean Max | |
|---|---|---|---|---|---|---|
| Test | Needle | Length | Tubing | Diameter | Pressure | Standard |
| Device | Gauge (Ga) | (mm) | Length (in) | (mm) | (mmHg) | Deviation |
| A | 27 | 12 | 24 | 0.4 | 956 | 36.6 |
| B | 27 | 12 | 24 | 0.5 | 672 | 0.4 |

10% human IgG (Carimune ™) (n = 2); Flow rate = 1.7 mL/min

TABLE 5
The Effect of Needle Shape on Inline Pressure

| | | | | | | Tubing | Mean | |
|---|---|---|---|---|---|---|---|---|
| | | | Needle | Needle | Tubing | Inner | Max | |
| Test | Needle | Needle | Gauge | Length | Length | Diameter | Pressure | Standard |
| Device | Shape | on/off | (Ga) | (mm) | (in) | (mm) | (mmHg) | Deviation |
| A | Sharp | On | 24 | 9 | 7 | 1 | BML | |
| | Bend | | | | | | | |
| B | Straight | On | 25 | 13 | 8 | 1.2 | 276 | 1.8 |
| C | Sharp | Off | 24 | 9 | 7 | 1 | 28 | 1.4 |
| | Bend | | | | | | | |

10% human IgG (Carimune ™) (n = 3); Flow rate = 5 mL/min
BML = Beyond Measurable Limits

TABLE 6
The Effect of Needle Shape on Inline Pressure

| | | | | | | Mean | |
|---|---|---|---|---|---|---|---|
| | | Needle | Needle | Tubing | Tubing Inner | Max | |
| Test | Needle | Gauge | Length | Length | Diameter | Pressure | Standard |
| Device | Shape | (Ga) | (mm) | (in) | (mm) | (mmHg) | Deviation |
| A | Soft | 25 | 6 | 10 | 1 | 316 | 2.8 |
| | Bend | | | | | | |
| B | Straight | 25 | 6 | 10 | 1 | 234 | 0.7 |

10% human IgG (Carimune ™) (n = 2); Flow rate = 4 mL/min

TABLE 7
The Effect of Needle Shape on Inline Pressure

| | | | | | | Mean | |
|---|---|---|---|---|---|---|---|
| | | Needle | Needle | Tubing | Tubing Inner | May | |
| Test | Needle | Gauge | Length | Length | Diameter | Pressure | Standard |
| Device | Shape | (Ga) | (mm) | (in) | (mm) | (mmHg) | Deviation |
| A | Soft | 25 | 6 | 10 | 1 | 1344 | 2.8 |
| | Bend | | | | | | |
| B | Straight | 25 | 6 | 10 | 1 | 1318 | 0.7 |

20% human IgG (Carimune ™) at 4.0 mL/min flow rate using analog gauge: Soft bend = 1344 mmHg vs. Straight = 1318 mmHg

TABLE 8
Infusion Set Parameter Analysis

| | | | | Tubing | Mean | |
|---|---|---|---|---|---|---|
| | Needle | Needle | Tubing | Inner | Max | |
| Infusion | Gauge | Length | Length | Diameter | Pressure | Standard |
| Set | (Ga) | (mm) | (in) | (mm) | (mmHg) | Deviation |
| A | 24 | 9 | 32 | 0.6 | BML | |
| B | 24 | 9 | 7 | 1.0 | 285 | 0.4 |
| C | 23 | 19 | 12 | 1.2 | 130 | 0.7 |
| D | 23 | 19 | 12 | 1.0 | 101 | 1.1 |
| E | 25 | 13 | 8 | 1.2 | 217 | 1.7 |
| F | 27 | 13 | 8 | 1.2 | BML | |
| G | 27 | 4 | 6 | 0.5 | BML | |
| H | 27 | 6 | 23 | 0.3 | BML | |

10% GammaGard ™, Flow rate 5 mL/minute

TABLE 9
Infusion Set Parameter Analysis

| | | | | Tubing | Mean | |
|---|---|---|---|---|---|---|
| | Needle | Needle | Tubing | Inner | Max | |
| Infusion | Gauge | Length | Length | Diameter | Pressure | Standard |
| Set | (Ga) | (mm) | (in) | (mm) | (mmHg) | Deviation |
| A | 9 | 32 | 0.6 | 0.6 | BML | |
| B | 9 | 7 | 1.0 | 1.0 | BML | |
| C | 19 | 12 | 1.2 | 1.2 | 408 | 11.5 |
| D | 19 | 12 | 1.0 | 1.0 | BML | 1.0 |
| E | 13 | 8 | 1.2 | 1.2 | BML | |

20% GammaGard ™, Flow rate 3 mL/minute
BML = Beyond Measurable Limit (>458 mmHg or >8.9 PSI); n = 3-6 replicates/test article

TABLE 10
The Effect of Needle Gauge on Inline Pressure

| | | | | Tubing | Mean | |
|---|---|---|---|---|---|---|
| | Needle | Needle | Tubing | Inner | Max | |
| Test | Gauge | Length | Length | Diameter | Pressure | Standard |
| Device | (Ga) | (mm) | (in) | (mm) | (mmHg) | Deviation |
| A | 23 | 19 | 12 | 1.0 | 49 | 0.2 |
| B | 25 | 19 | 12 | 1.0 | 108 | 0.5 |

Sterile Water

TABLE 11
The Effect of Needle Length on Inline Pressure

| | | | | Tubing | Mean | |
|---|---|---|---|---|---|---|
| | Needle | Needle | Tubing | Inner | Max | |
| Test | Gauge | Length | Length | Diameter | Pressure | Standard |
| Device | (Ga) | (mm) | (in) | (mm) | (mmHg) | Deviation |
| A | 24 | 9 | 32 | 0.6 | 216 | 5.0 |
| B | 24 | 6 | 32 | 0.6 | 210 | 7.1 |

Sterile Water

TABLE 12
The Effect of Tubing Length on Inline Pressure

| | | | | Tubing | Mean | |
|---|---|---|---|---|---|---|
| | Needle | Needle | Tubing | Inner | Max | |
| Test | Gauge | Length | Length | Diameter | Pressure | Standard |
| Device | (Ga) | (mm) | (in) | (mm) | (mmHg) | Deviation |
| A | 23 | 19 | 12 | 1.0 | 49 | 0.2 |
| B | 23 | 19 | 7 | 1.0 | 43 | 0.7 |

Sterile Water

TABLE 13
The Effect of Tubing Inner Diameter on Inline Pressure

| | | | | Tubing | Mean | |
|---|---|---|---|---|---|---|
| | Needle | Needle | Tubing | Inner | Max | |
| Test | Gauge | Length | Length | Diameter | Pressure | Standard |
| Device | (Ga) | (mm) | (in) | (mm) | (mmHg) | Deviation |
| A | 24 | 9 | 32 | 0.6 | 216 | 5.0 |
| B | 24 | 9 | 7 | 1 | 99 | 0.4 |

Sterile Water

TABLE 14
The Effect of Needle Shape on Inline Pressure

| | | | | | | Tubing | Mean | |
|---|---|---|---|---|---|---|---|---|
| | | | Needle | Needle | Tubing | Inner | Max | |
| Test | Needle | Needle | Gauge | Length | Length | Diameter | Pressure | Standard |
| Device | Shape | on/off | (Ga) | (mm) | (in) | (mm) | (mmHg) | Deviation |
| A | Bent | On | 24 | 9 | 7 | 1 | BML | |
| B | Straight | On | 25 | 13 | 8 | 1.2 | 276 | 1.8 |
| C | Bent | Off | 24 | 9 | 7 | 1 | 28 | 1.4 |

10% human IgG (Carimune ™) (n = 3 replicates/test article)
BML = Beyond Measurable Limit (>458 mmHg or >8.9 PSI)

### EXAMPLE 2

### Flow Analysis of Infusion Sets

This example illustrates flow rate characteristics for a variety of infusion set designs. The experiment was carried out using the same methods as described in Example 1 utilizing infusion sets with the features as presented in Table 15.

TABLE 15
Analysis of Custom Designed Infusion Sets

| | | | | Tubing | |
|---|---|---|---|---|---|
| | | | Tubing | Inner | |
| Infusion | Needle Gauge | Needle Length | Length | Diameter | Needle |
| Set | (Ga) | (mm) | (in) | (mm) | Shape |
| A* | 27 | 13 | 8 | 1-1.2 | Straight |
| B* | 25 | 13 | 8 | 1-1.2 | Straight |
| C | 27 | 6 | 10 | 1 | Soft |
| | | | | | Curve |
| D | 27 | 6 | 10 | 1 | Straight |
| E | 25 | 6 | 10 | 1 | Soft |
| | | | | | Curve |
| F | 25 | 6 | 10 | 1 | Straight |
| G | 24 | 6 | 10 | 1 | Soft |
| | | | | | Curve |
| H | 24 | 6 | 10 | 1 | Straight |

*Denotes non-custom infusion sets used for reference.

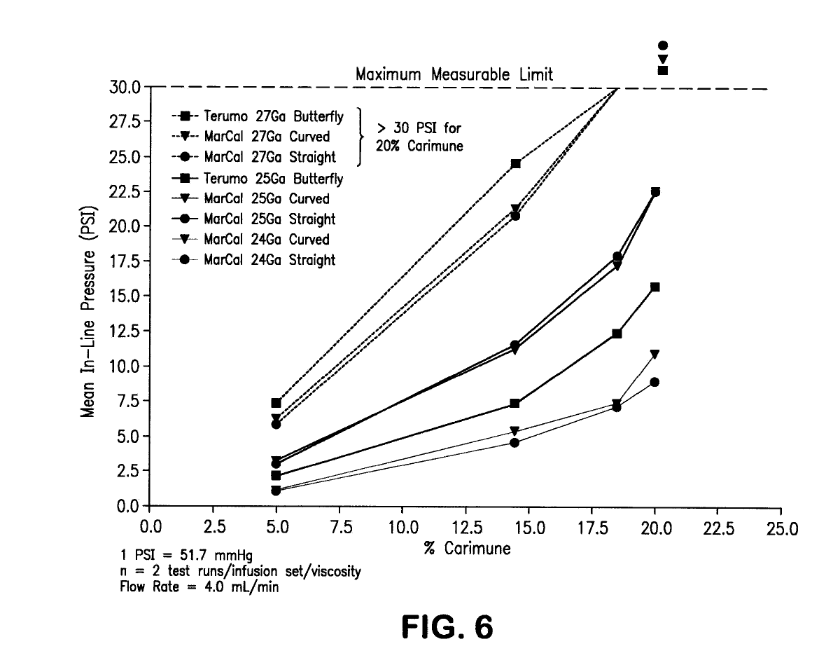

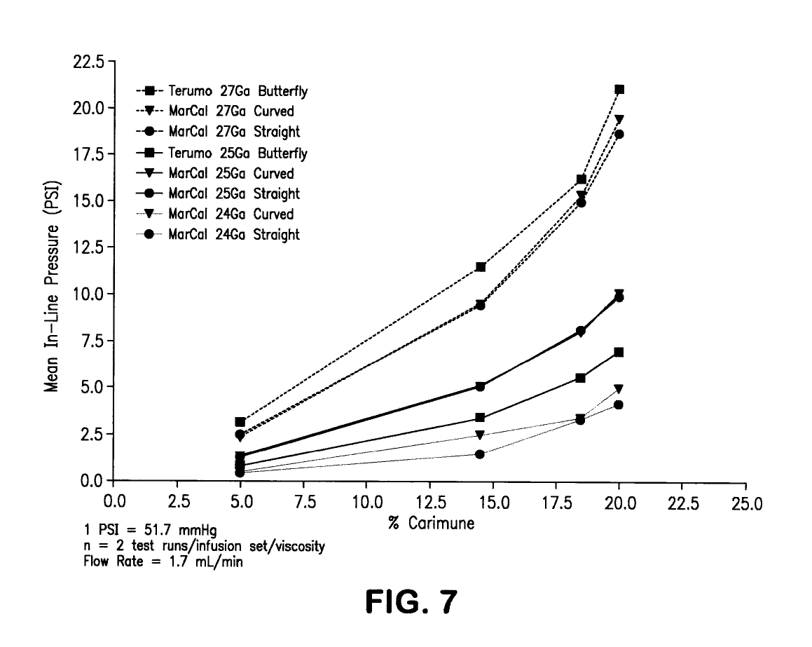

The results of flow analysis using different percentages of human IgG (Carimune™) solution at two different flow rates using the infusion set designs presented in Table 14 are shown in FIGS. 6 and 7.

FIG. 6 is a graphical representation of inline pressures generated by the various infusion sets represented in Table 15. Solutions containing different percentages of human IgG (Carimune™) were used at a flow rate of 4.0 ml/min. The top set of three lines presented in the plot correspond to the infusion sets having 27 Ga needles. The middle set of three lines presented in the plot correspond to the infusion sets having 25 Ga needles. The bottom set of 2 lines presented in the plot correspond to the infusion sets having 24 Ga needles.

FIG. 7 is a graphical representation of inline pressures generated by the various infusion sets represented in Table 15. Solutions containing different percentages of human IgG (Carimune™) were used at a flow rate of 1.7 ml/min. The top set of three lines presented in the plot correspond to the infusion sets having 27 Ga needles. The middle set of three lines presented in the plot correspond to the infusion sets having 25 Ga needles. The bottom set of 2 lines presented in the plot correspond to the infusion sets having 24 Ga needles.

Although the invention has been described with reference to the above examples, it will be understood that modifications and variations are encompassed within the spirit and scope of the invention. Accordingly, the invention is limited only by the following claims.

What is claimed is:

1. A subcutaneous infusion system comprising:
a) a needle of 24 to 27 Ga comprising a shaft having an internal duct of unvarying diameter defining a fluid pathway between openings at distal and proximal ends of the needle, and wherein a hub in fluid communication with the duct is disposed at the distal end and the needle length therefrom to the proximal end is about 4 to 6 mm; and
b) a medication dispensing device in detachable fluid communication with the needle duct, the device containing about 3 to 100 mls of a highly viscous therapeutic fluid composition, wherein the system is configured for subcutaneous delivery of the therapeutic fluid composition at a flow rate of about 1-20 mls per minute.

2. The infusion system of claim 1, wherein the dispensing device is attachable to the hub for indirect fluid communication with the needle duct.

3. The infusion system of claim 1, wherein the needle and delivery device are in fluid communication via tubing connecting the distal opening of the needle to the dispensing device.

4. The infusion system of claim 3, wherein the tubing has an internal diameter of about 1-1.2 mm.

5. The infusion system of claim 4, wherein the tubing has an internal diameter of about 1 mm.

6. The infusion system of claim 3, wherein the tubing has a length of about 6 to 10 inches.

7. The infusion system of claim 1, wherein the needle is straight.

8. The infusion system of claim 1, wherein the needle has a bent shaft.

9. The infusion system of claim 8, wherein the shaft is bent 90 degrees.

10. The infusion system of claim 1, wherein the delivery device includes about 3 to 15 mls of a therapeutic fluid composition.

11. The infusion system of claim 10, wherein the delivery device includes about 10 to 15 mls of a therapeutic fluid composition.

12. The infusion system of claim 1, wherein the therapeutic fluid composition has a viscosity of about 10 to 20 cP.

13. The infusion system of claim 1, wherein the dispensing device comprises a fluid reservoir.

14. The infusion system of claim 13, wherein the dispensing device is a syringe.

15. The infusion system of claim 14, wherein the syringe is automated for constant fluid delivery.

16. The infusion system of claim 13, wherein the fluid reservoir is a pre-filled cartridge.

17. The infusion system of claim 16, wherein the fluid reservoir is pressurized.

18. The infusion system of claim 1, wherein the needle comprises any number greater than 1 opening or hole disposed on the side of each needle shaft up to as many as can be added without impairing rigidity or structural integrity of the needle shaft.

19. The infusion system of claim 1, wherein the system further comprises additional needles of 24 to 27 Ga, wherein each additional needle comprises a second shaft having an internal duct of unvarying diameter defining a fluid pathway between openings at distal and proximal ends of each needle; and further wherein the hub is also in fluid communication with at least one additional needle and each additional needle has a length of about 4 to 6 mm measured from the hub to the proximal opening of the second needle.

20. The infusion system of claim 19, wherein the first and additional needles are the same gauge.

21. The infusion system of claim 19, wherein each needle comprises any number greater than 1 opening or hole disposed on the side of each needle shaft up to as many as can be added without impairing rigidity or structural integrity of the needle shaft.

22. The infusion system of claim 19, wherein the system comprises one additional needle.

23. The infusion system of claim 1, wherein the system is configured for subcutaneous delivery of the therapeutic fluid composition at a flow rate of about 3-5 ml/min.

24. A method for subcutaneous delivery of a high viscosity therapeutic fluid composition to a subject in need thereof comprising administering to the subject a therapeutic fluid using the device of claim 1, wherein the therapeutic fluid is administered at a flow rate of about 1-20 ml/min.

25. The method of claim 24, wherein the therapeutic fluid is administered at a flow rate of about 3-5 ml/min.

26. The method of claim 24, wherein the therapeutic fluid has a viscosity of about 10 to 20 cP.

27. The method of claim 24, wherein the flow rate is greater than about 10 ml/min and the therapeutic fluid has a viscosity less than about 5 cP.

28. The method of claim 24, wherein the therapeutic fluid is administered through the dermis of the abdomen, arm or thigh.

29. The method of claim 24, wherein the therapeutic fluid comprises a hyaluronidase enzyme.

30. The method of claim 29, wherein the therapeutic fluid comprises a hyaluronidase is a human hyaluronidase.

31. The method of claim 30, wherein the therapeutic fluid comprises a chemotherapeutic agent.

32. A method for subcutaneous delivery of a high volume of a therapeutic fluid to a subject in need thereof comprising administering to the subject a therapeutic fluid using a subcutaneous infusion system comprising:
a) a needle of 18 Ga comprising a shaft having an internal duct of unvarying diameter defining a fluid pathway between openings at distal and proximal ends of the needle, and wherein a hub in fluid communication with the duct is disposed at the distal end and the needle length therefrom to the proximal end is about 4 to 6 mm; and
b) a medication dispensing device in detachable fluid communication with the needle duct, the device containing about 3 to 100 mls of a highly viscous therapeutic fluid composition,
wherein the fluid is administered at a flow rate greater than about 20 ml/min.

33. The infusion system of claim 32, wherein the needle comprises any number greater than 1 opening or hole disposed on the side of each needle shaft up to as many as can be added without impairing rigidity or structural integrity of the needle shaft.

34. The method of claim 32, wherein the system further comprises additional needles, wherein each additional needle comprises a second shaft having an internal duct of unvarying diameter defining a fluid pathway between openings at distal and proximal ends of each needle; and further wherein the hub is also in fluid communication with at least one additional needle and each additional needle has a length of about 4 to 6 mm measured from the hub to the proximal opening of the second needle.

35. The infusion system of claim 34, wherein each needle comprises any number greater than 1 opening or hole disposed on the side of each needle shaft up to as many as can be added without impairing rigidity or structural integrity of the needle shaft.
